(19) United States
(12) Patent Application Publication
(10) Pub. No.: US 2025/0308039 A1
(43) Pub. Date: Oct. 2, 2025

(54) TELEHEALTH SMART RAILS SYSTEM

(71) Applicant: LG ELECTRONICS INC., Seoul (KR)

(72) Inventors: Davide DI CENSO, Oakland, CA (US); Alicia STICE, Tulsa, OK (US); Prathamesh VASTE, Santa Clara, CA (US); Joshua MILBURN, Roanoke, VA (US)

(73) Assignee: LG ELECTRONICS INC., Seoul (KR)

(21) Appl. No.: 19/091,510

(22) Filed: Mar. 26, 2025

Related U.S. Application Data
(60) Provisional application No. 63/571,391, filed on Mar. 28, 2024.

(51) Int. Cl. G06T 7/246; G06T 7/73; G16H 40/20

(52) U.S. Cl. CPC G06T 7/246; G06T 7/73; G16H 40/20; G06T 2207/10016; G06T 2207/20081; G06T 2207/30004; G06T 2207/30196; G06T 2207/30232

(57) ABSTRACT

According to at least one embodiment, a method of monitoring movement of a skeleton of at least a first person located in a healthcare setting includes: receiving first coordinate information identifying a selected periphery of a portion of a displayed video image depicting the healthcare setting; and receiving second coordinate information identifying locations of a plurality of skeletal keypoints of the first person. The method further includes: based on the second coordinate information, identifying at least a first skeletal segment defined by a first pair of skeletal keypoints; tracking coordinates identifying the locations of the first pair of skeletal keypoints over a plurality of successive video images; determining that the selected periphery is crossed by detecting an intersection between the selected periphery and the first skeletal segment; and in response to determining that the selected periphery is crossed, transmitting a message indicating the first person has crossed the selected periphery.

### CROSS-REFERENCE TO RELATED APPLICATION(S)

Pursuant to 35 U.S.C. § 119(e), this application claims the benefit of U.S. Provisional Patent Application No. 63/571,391, filed Mar. 28, 2024, the contents of which are hereby incorporated by reference herein in its entirety.

### BACKGROUND

Annually across the United States, falls account for over $34 billion in healthcare costs.

Each year, 700,000 to 1,000,000 patients fall in U.S. hospitals. Each fall costs hospitals an average of $64,526, where the level of injury is not significantly associated with cost.

Similarly, elopement by patients who are a flight risk (e.g., who might run away, or leave without notice) also incurs high costs to hospitals. This is a risk that carries not only financial consequences for the hospital, but also a level of danger with respect to patients' health outcome.

Therefore, steps should be taken to reduce the likelihood of falls and attempted elopement.

### SUMMARY

Aspects of this disclosure are directed toward assisting healthcare professionals (e.g., patient safety monitoring (PSM) professionals) to monitor patients more effectively, in order to improve patient safety. For example, certain aspects are directed to providing notifications to such professionals upon detection of movements and/or actions that can be dangerous, e.g., with respect to fall and/or flight risk. Such movements and/or actions may be made or taken by a patient himself, or by another person who is near the patient (e.g., a person who may be attempting to physically interact with the patient).

According to aspects of this disclosure, a visual computer-implemented tool is used to establish (or define) one or more virtual boundaries in a monitored healthcare setting, for improving patient safety. The tool may be used by PSM professionals (e.g., eSitters) who monitor patients and respond to potential safety risks. Aspects of this disclosure are directed toward enabling such professionals to perform reliable, real-time monitoring and toward providing such professionals with alert notifications based on the established virtual boundaries. Aspects of this disclosure are directed to improving the quality of monitoring by such professionals, e.g., by increasing the number of patients each PSM professional can monitor effectively at a given time.

According to at least one embodiment, a method of monitoring movement of a skeleton of at least a first person located in a healthcare setting includes: receiving first coordinate information identifying a selected periphery of a portion of a displayed video image, the displayed video image depicting the healthcare setting; and receiving second coordinate information identifying locations of a plurality of skeletal keypoints of the first person located in the healthcare setting. The method further includes: based on the second coordinate information, identifying at least a first skeletal segment, the first skeletal segment defined by a first pair of skeletal keypoints of the plurality of skeletal keypoints; tracking coordinates identifying the locations of the first pair of skeletal keypoints over a plurality of successive video images depicting the healthcare setting; based on the first coordinate information, determining that the selected periphery is crossed by detecting an intersection between the selected periphery and the first skeletal segment; and in response to determining that the selected periphery is crossed, transmitting a message indicating that the first person located in the healthcare setting has crossed the selected periphery.

According to at least one embodiment, an artificial intelligence (AI) device is configured to monitor movement of a skeleton of at least a first person located in a healthcare setting. The AI device includes: at least one transceiver; and at least one processor. The at least one processor is configured to: receive first coordinate information identifying a selected periphery of a portion of a displayed video image, the displayed video image depicting the healthcare setting; receive second coordinate information identifying locations of a plurality of skeletal keypoints of the first person located in the healthcare setting; based on the second coordinate information, identify at least a first skeletal segment, the first skeletal segment defined by a first pair of skeletal keypoints of the plurality of skeletal keypoints; track coordinates identifying the locations of the first pair of skeletal keypoints over a plurality of successive video images depicting the healthcare setting; based on the first coordinate information, determine that the selected periphery is crossed by detecting an intersection between the selected periphery and the first skeletal segment; and in response to determining that the selected periphery is crossed, transmit a message indicating that the first person located in the healthcare setting has crossed the selected periphery.

According to at least one embodiment, a non-transitory storage medium stores instructions that, when executed, cause at least one processor to perform operations. The operations include: receiving first coordinate information identifying a selected periphery of a portion of a displayed video image, the displayed video image depicting a healthcare setting; receiving second coordinate information identifying locations of a plurality of skeletal keypoints of a first person located in the healthcare setting; based on the second coordinate information, identifying at least a first skeletal segment, the first skeletal segment defined by a first pair of skeletal keypoints of the plurality of skeletal keypoints; tracking coordinates identifying the locations of the first pair of skeletal keypoints over a plurality of successive video images depicting the healthcare setting; based on the first coordinate information, determining that the selected periphery is crossed by detecting an intersection between the selected periphery and the first skeletal segment; and in response to determining that the selected periphery is crossed, transmitting a message indicating that the first person located in the healthcare setting has crossed the selected periphery.

### BRIEF DESCRIPTION OF THE DRAWINGS

FIG. 1 illustrates an example establishment of virtual boundaries according to at least one embodiment.

FIG. 2 illustrates an example of inside-out tracking (inside-out boundary crossing) according to at least one embodiment.

FIG. 3 describes an example of outside-in tracking (outside-in boundary crossing) according to at least one embodiment.

FIG. 4A illustrates an example of a silhouette of a human that appears in a video image that is analyzed.

FIG. 4B illustrates example keypoints identified with respect to an arrangement (the silhouette of FIG. 4A).

FIG. 5 illustrates examples of kept keypoints and identified segments between pairs of kept keypoints.

FIG. 6 illustrates intersection of a segment and a particular boundary at a particular moment in time.

FIG. 7A illustrates a scenario in which certain skeletal segments of a person are determined to have crossed more than one boundary according to at least one embodiment.

FIG. 7B illustrates particular crossings or intersections with reference to the scenario illustrated in FIG. 7A.

FIG. 8A illustrates an example of a set of skeletal segments corresponding to a highest (or high) sensitivity setting according to at least one embodiment.

FIG. 8B illustrates an example of a set of skeletal segments corresponding to a medium (or intermediate) sensitivity setting according to at least one embodiment.

FIG. 8C illustrates an example of a set of skeletal segments corresponding to a low (or lowest) sensitivity setting according to at least one embodiment.

FIG. 9 illustrates a flowchart of a method of monitoring movement of a skeleton of at least a first person located in a healthcare setting according to at least one embodiment.

FIG. 10 is a block diagram of an artificial intelligence (AI) device according to at least one embodiment.

FIG. 11 is a diagram illustrating a system in which an AI device and devices operable by one or more PSM professionals are linked according to at least one embodiment.

### DETAILED DESCRIPTION

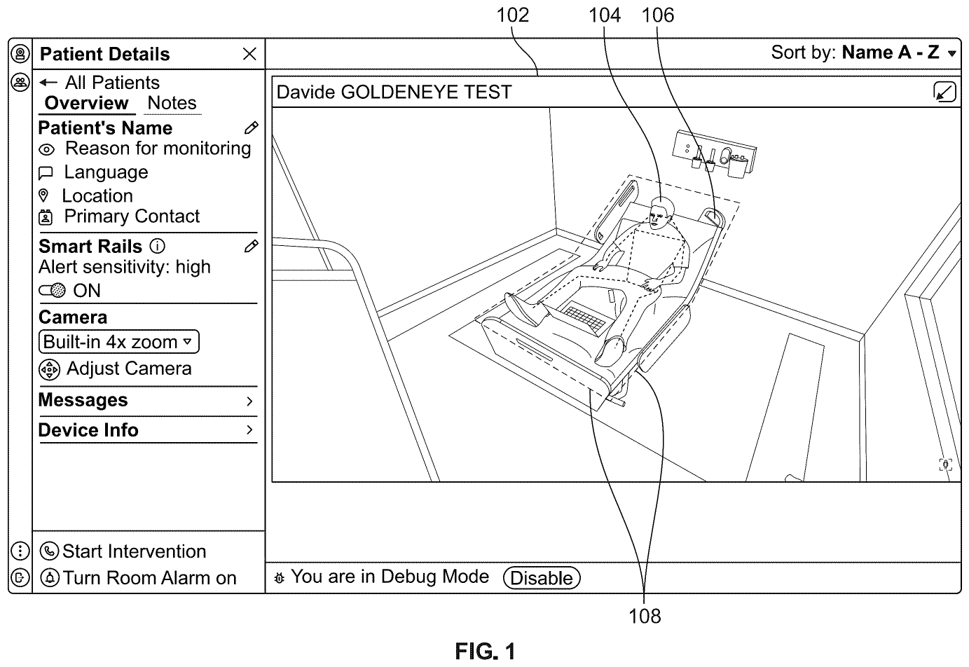

FIG. 1 illustrates an example establishment of virtual boundaries according to at least one embodiment. With reference to FIG. 1, display of a video image 102 is provided. The display may be provided at a display device (e.g., a video monitor) that is utilized by one or more patient safety monitoring (PSM) professionals. The video image 102 may be captured by one or more cameras that are positioned in a healthcare setting such as a hospital, a nursing facility, etc. For example, the camera(s) may be positioned in the room of a medical patient. In the example of FIG. 1, the video image 102 captures a human patient 104 who is positioned on a bed 106.

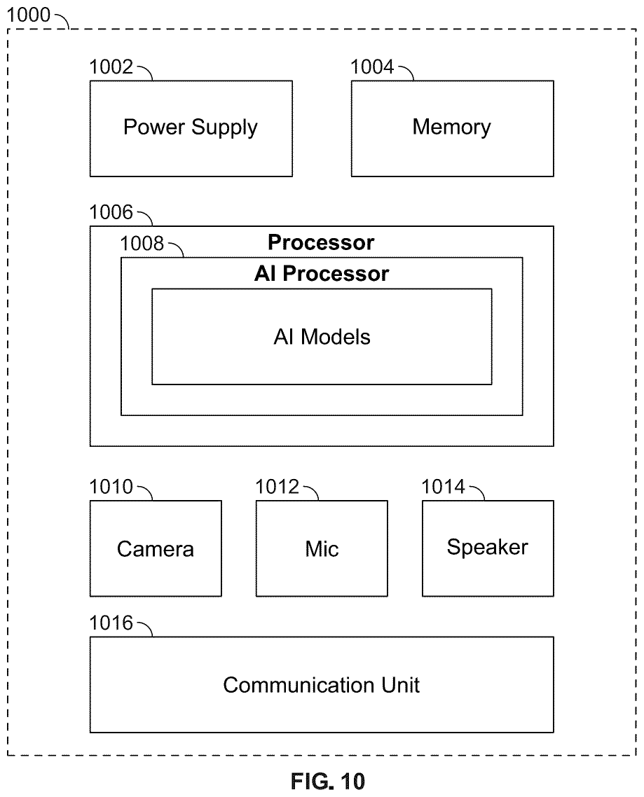

According to at least one embodiment, based on the display of the video image 102, the PSM professional may operate a visual computer-implemented tool implemented in a device, e.g., device 1000, which will described in more detail later with reference to FIG. 10. The PSM professional may operate the tool to define one or more virtual boundaries 108. For example, the virtual boundaries 108 may be defined to form a closed polygon having continuous edges. In the example of FIG. 1, the virtual boundaries 108 form a closed polygon having six continuous edges and six vertices.

According to at least one embodiment, the virtual boundaries 108 may be selected by the PSM professional to enclose a specific portion of the video image 102. According to at least one alternative embodiment, the virtual boundaries 108 are positioned by an analytical model that will be described later herein. For example, such an analytical model may identify edges of a piece of furniture (e.g., a bed or a chair) and position the virtual boundaries 108 at such edges. As another example, the analytical model may position virtual boundaries 108 around an identified person.

In the example of FIG. 1, the virtual boundaries 108 are selected to enclose a portion of the video image 102 in which the human patient 104 and the bed 106 are depicted. It is understood that the virtual boundaries 108 may be selected to, either alternatively or in addition, enclose a different piece of furniture. For example, the virtual boundaries 108 may be selected to enclose a chair that is used by the human patient 104 and that is also captured in the video image 102. Also, it is understood that the virtual boundaries 108 may be selected to, either alternatively or in addition, enclose an unfurnished area of the patient's room (e.g., an area of the room that is at or around the entry/exit door).

By defining the virtual boundaries 108 as described above, the PSM professional effectively selects an area of the setting in which the human patient 104 (or another person) may move about (e.g., an area at or around the patient's bed or chair, an area at or adjacent to the entry/exit door, etc.). Conversely, virtual boundaries 108 may be used to define an area into which the human patient 104 is to be excluded from entering. As will be described in more detail below with reference to various embodiments, the periphery (or boundary) of such a selected area is effectively monitored for movement and/or crossing of the periphery by one or more persons.

For example, with reference to FIG. 1, the periphery of the area of the healthcare setting that corresponds to the portion of the video image 102 enclosed by virtual boundaries 108 is effectively monitored for movement and/or crossing of the periphery by one or more persons (e.g., human patient 104). Upon detection of such movement and/or crossing, an electronic notification (e.g., audio and/or visual) may be provided to the PSM professional. As such, the PSM professional is aided in monitoring for occurrence of potentially hazardous situations. This assists the PSM professional in monitoring the human patient 104 more effectively, in order to improve patient safety.

According to various embodiments, virtual boundaries can be effectively monitored for movement and/or crossing of the periphery in different directional situations.

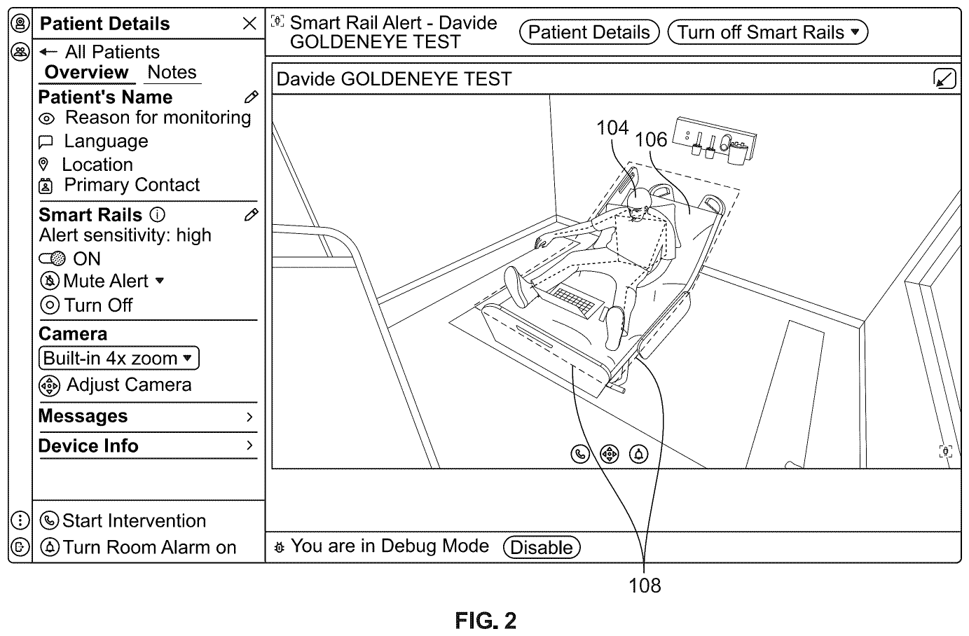

For example, FIG. 2 illustrates an example of inside-out tracking according to at least one embodiment. In the example illustrated, the human patient 104 extends his right forearm during the act of attempting to arise from the bed 106. In this situation, the movement (of the right forearm) starting from inside the periphery of the area and continuing past the virtual boundary 108 is tracked (or detected).

Inside-out tracking may be useful in improving prevention of falls. Patients at risk of falls may be required to remain in bed or seated during periods when assistance by another human is not available. While attempting to stand, such a patient may effectively cross virtual boundaries (e.g., virtual boundaries 108) that had been defined earlier. Detection of such a crossing would cause a notification to be provided to a PSM professional. Such an individual may, in turn, improve patient safety by drawing the attention of the PSM professional towards the video feed showing that the patient is crossing the virtual boundaries, notifying on-site staff and/or sending a nurse to assist the patient.

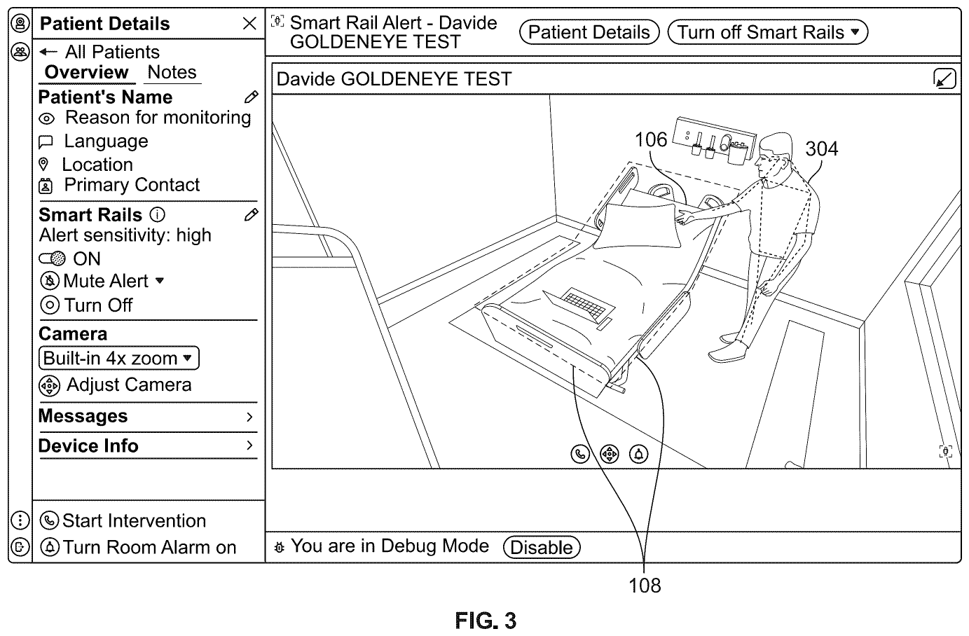

FIG. 3 describes an example of outside-in tracking according to at least one embodiment. In the example illustrated, a second person 304 (e.g., a person different from the patient 104 of FIG. 2) extends his right forearm during the act of attempting to physically interact with a patient positioned on the bed 106. In this situation, the movement (of the right forearm) starting from outside the periphery of the area and continuing past the virtual boundary 108 is tracked (or detected).

Outside-in tracking may be useful in detecting potentially dangerous physical interactions with the patient by another person. Such other person may be a visitor or a healthcare worker who physically moves too close to the patient, thereby posing potential risks to the safety of the patient. Detection of such a crossing would cause a notification to be provided to a PSM professional. Such an individual may, in turn, improve patient safety, e.g., by supervising the situation remotely and responding as appropriate or necessary.

Outside-in tracking may be useful in lowering the risk of flight or elopement. For example, a patient who is deemed to be a flight risk may attempt to leave his room by leaving through the exit door. As described earlier, virtual boundaries such as virtual boundaries 108 may be defined to enclose an area of the room that is at or around the entry/exit door. As the patient approaches the exit door in an attempt to leave, detection of a crossing of such virtual boundaries (by the body of the patient) may occur. Detection of such a crossing would cause a notification to be provided to a PSM professional.

As noted earlier, boundaries may be selected with reference to a display of a video image (e.g., video image 102). With reference back to FIG. 1, an individual (e.g., a PSM professional) may select boundaries 108 defining a closed polygon. For example, the closed polygon may have six vertices (e.g., hexagonal). However, it is understood that the closed polygon may be defined to have fewer or more indices.

Coordinates corresponding to the boundaries (e.g., locational coordinates of the vertices of the polygon) may be used for detecting crossings of one or more of the boundaries. For example, a device according to at least one embodiment receives relative polygon coordinates for the video feed.

In this regard, the video feed seen by the PSM professional may be displayed in a web app, which is aware of the resolution (e.g., video resolution of the feed), as well as the x-y “mouse” coordinates of each “click” made by the PSM professional upon moving (changing) the vertices.

The web app calculates the relative coordinates, e.g., on a 0-1 scale, where a y-coordinate value of ‘0’ denotes ‘top’ in the Y axis, an x-coordinate value of ‘0’ denotes ‘left’ in the X axis, a y-coordinate value of ‘1’ denotes ‘bottom’ in the Y axis, and an x-coordinate value of ‘1’ denotes ‘right’ in the X plane. As such, the (x, y) coordinate pair (0, 0) would denote ‘uppermost left pixel’, and the (x, y) coordinate pair (1, 1) would denote ‘lowermost right pixel’.

In this way, the fractional (0->1, or 0-100%) coordinates of each vertex are computed in the web app, and sent to the application running on the device.

The application on the device can then apply these same coordinates to determine coordinates of the boundaries on the video feed it is processing, for example, by multiplying the fractional value by the resolution. This is because the video source of the video feed viewed by the PSM professional and the video source of the video feed being analyzed by the device are the same (e.g., a camera positioned in the room of a medical patient). Relative coordinates are used to overcome technical limitations of web technologies (WebRTC) in order to map the video resolution captured on the patient side device to the displayed video resolution on the monitor viewed by the PSM professional.

The device may also receive information indicating a resolution of the video feed from which the video image was taken. The relative coordinates may be mapped to the feed resolution, in order to determine proper reference coordinates corresponding to the boundaries.

According to one or more embodiments, one or more video images of a video feed are analyzed by an analytical model (e.g., a model that is driven by artificial intelligence (AI)). For example, a video image similar to the video image 102 of FIG. 1 may be analyzed. According to one or more embodiments, the analytical model may be included (or implemented) in the device. Alternatively, the analytical model may be external to the device. As will be described in more detail below, the analytical model processes the video image to identify the presence of any humans who appear in the image.

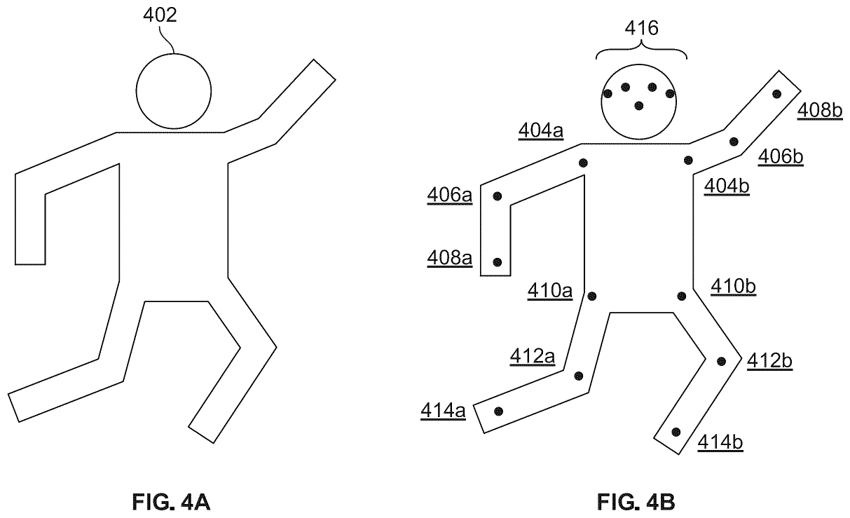

FIG. 4A illustrates an example of a silhouette 402 of a human that appears in a video image that is analyzed by the model. According to at least one embodiment, the model analyzes the image to identify particular keypoints (see, e.g., FIG. 4B). As described herein with reference to various embodiments, such keypoints may serve as main points tracked by the device (e.g., analytical model). These points may be tracked for purposes of detecting boundary crossing and may be used for defining sensitivity levels

Such keypoints may correspond to particular skeletal features. For example, with reference to the silhouette 402 of FIG. 4A, such keypoints may include the keypoints of the shoulders 404a, 404b, the elbows 406a, 406b, the wrists 408a, 408b, the hips 410a, 410b, the knees 412a, 412b, and the ankles 414a, 414b, etc. The keypoints may also include features of the skull, including keypoints 416 corresponding to the eyes and the nose. According to at least one embodiment, the presence of a human in the video image is identified based on the identification of such keypoints in an arrangement such as the silhouette 402.

According to at least one embodiment, the model is (or may include) a convolutional neural network. Such a neural network may serve as a backbone feature extractor. Additional convolutional layers on top of the backbone feature extractor may serve to generate heatmaps to locate keypoints such as the keypoints described earlier with reference to FIG. 4B.

The model may output coordinates corresponding to the keypoints. For example, such coordinates may be locational coordinates with respect to the video image in which the keypoints were identified. The device receives such relative keypoint coordinates for the video feed. This addresses situations in which the resolution of video captured by a camera (e.g., 720p) for the model is scaled down to a lower resolution (e.g., 280p) for performance reasons. Keypoints generated in the lower resolution would then be converted into the higher resolution (e.g., 720p) in order to correctly apply the mathematical equation to detect boundary crossing.

According to at least one embodiment, the coordinates of a given keypoint may be accompanied by probability data indicating a level of confidence that the model has with respect to accuracy of the identification of that particular keypoint. By way of example, the probability data (or particular ranges thereof) may indicate high level of confidence, intermediate level of confidence, low level of confidence, etc.

If the model identifies the presence of more than one human in a single video image, then multiple sets of data may be output. For example, for each human that is identified, the model may provide an array that includes the coordinates of the corresponding keypoints. Such keypoints may be accompanied by the corresponding probability data.

Based on the data received from the model, the device may identify segments that are defined by the keypoints. These segments may correspond to specific skeletal segments (or fragments).

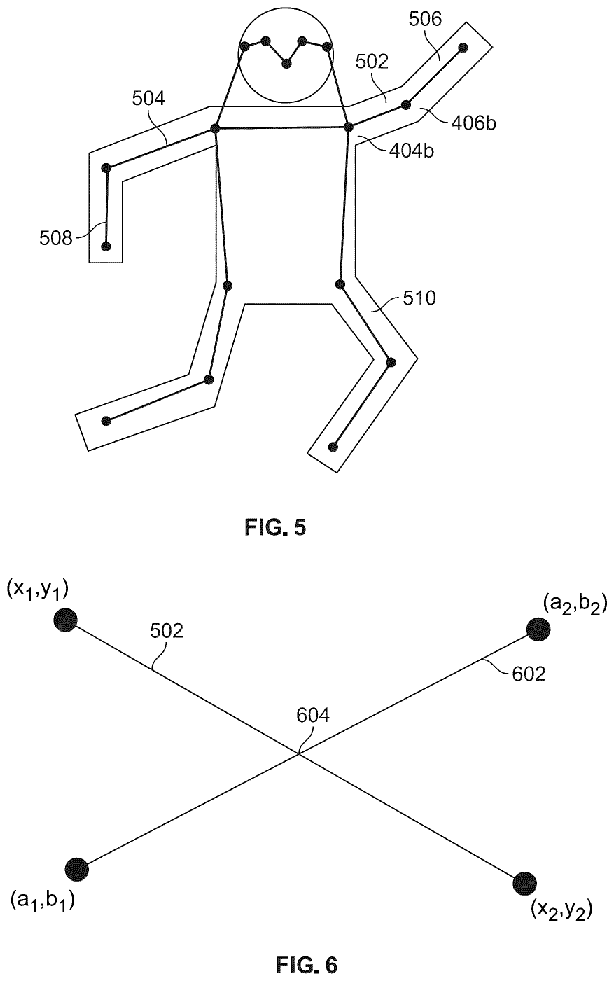

According to at least one embodiment, the device filters the received coordinate data based on the corresponding probability data. For example, only keypoints having probability data that meet at least a particular confidence threshold (e.g., a threshold value indicating a high level of confidence) are kept for further processing. In contrast, keypoints not meeting such a threshold may be disregarded. The keypoints that are kept may then be used to identify specific segments (e.g., between specific pairs of keypoints). This will be described in more detail with reference to FIG. 5.

FIG. 5 illustrates example keypoints that are kept, e.g., as a result of filtering. In the example of FIG. 5, the kept keypoints include all of the keypoints illustrated in the example of FIG. 4B. However, it is understood that the number of kept keypoints may be fewer than all of the keypoints that had been identified by the model.

With reference to FIG. 5, the kept keypoints include the keypoint of the shoulder 404b corresponding to the left shoulder and the keypoint of the elbow 406b corresponding to the left elbow. With respect to the keypoints of the shoulder 404b and the elbow 406b, a segment 502 that extends between this pair of keypoints is identified. The segment 502 may be labeled, by way of example, as “upper left arm.”

If the kept keypoints include all of the keypoints illustrated in the example of FIG. 4B, then a total of 18 distinct segments may be identified (see, e.g., the straight-line segments illustrated in FIG. 5.) The identified segments may include, for example, “upper left arm” segment 502, “upper right arm” segment 504, “lower left arm” segment 506, “lower right arm” segment 508, “upper left leg” segment 510, etc.

As will be explained in more detail below with reference to various embodiments, coordinates (e.g., tracked coordinates) of keypoints associated with one or more target segments are used to detect intersections of the segment with a particular boundary (e.g., a virtual boundary 108 as described earlier with reference to FIG. 1). In this manner, it is determined whether a human has crossed the particular boundary.

For purposes of description, an example determination of whether a particular segment has crossed one or more boundaries will now be described with reference to the “upper left arm” segment 502. It is understood that such description also applies toward determination of whether any other identified segment has crossed one or more boundaries.

It is understood that the coordinates corresponding to the “upper left arm” segment 502 do not necessarily remain constant across successive video images of a video feed. Rather, the coordinates will necessarily change as the location of the upper left arm of the person moves over time. Therefore, according to the at least one embodiment, the coordinates of the “upper left arm” segment 502 will be tracked and calibrated (e.g., updated) over successive video images (e.g., images following, or subsequent to, the video image 102 in the video feed). Here, frame interpolation and/or smoothing may be used in analyzing video images to address temporal instability, as will be described in more detail later. As the coordinates are tracked, it may be determined whether the segment 502 has crossed one or more boundaries.

FIG. 6 illustrates the “upper left arm” segment 502 and a particular boundary 602 of the closed polygon at a particular moment in time (i.e., in a particular video image or video frame). The coordinates (x₁, y₁) and (x₂, y₂) correspond to endpoints of the “upper left arm” segment 502. The coordinates (a₁, b₁) and (a₂, b₂) correspond to endpoints of the boundary 602.

According to at least one embodiment, the presence of a potential intersection of the “upper left arm” segment 502 with the boundary 602 is calculated based on the following expression:

$$( {x}_{1} - {x}_{2} ) ( {b}_{1} - {b}_{2} ) - ( {y}_{1} - {y}_{2} ) ( {a}_{1} - {a}_{2} ) .$$

If the above expression is calculated as having a value of 0, then it is determined that the “upper left arm” segment 502 does not intersect the boundary 602.

In contrast, if the above expression is calculated as having a non-zero value, then it is determined whether the segment 502 and the boundary 602 are in range of each other, e.g., whether they would intersect each other without extending into infinity. If it is determined that the segment 502 and the boundary 602 are in range of each other, then it is determined that the “upper left arm” segment 502 does intersect the boundary 602. Therefore, it is determined that a crossing of the boundary 602 has occurred (see, e.g., intersection 604 of FIG. 6).

The described determination may be performed with respect to each boundary (e.g., edge) of the polygon defined by the boundaries. For example, as described earlier with reference to FIG. 1, a polygon may be defined to have a total of 6 edges. In such a situation, determinations as to whether the “upper left arm” segment 502 has crossed each of the six edges may be made individually, in order to determine whether any of the edges have been crossed by the segment.

In one example, the described determination may be performed for all skeletal segments that were identified (e.g., all 18 segments illustrated in FIG. 5).

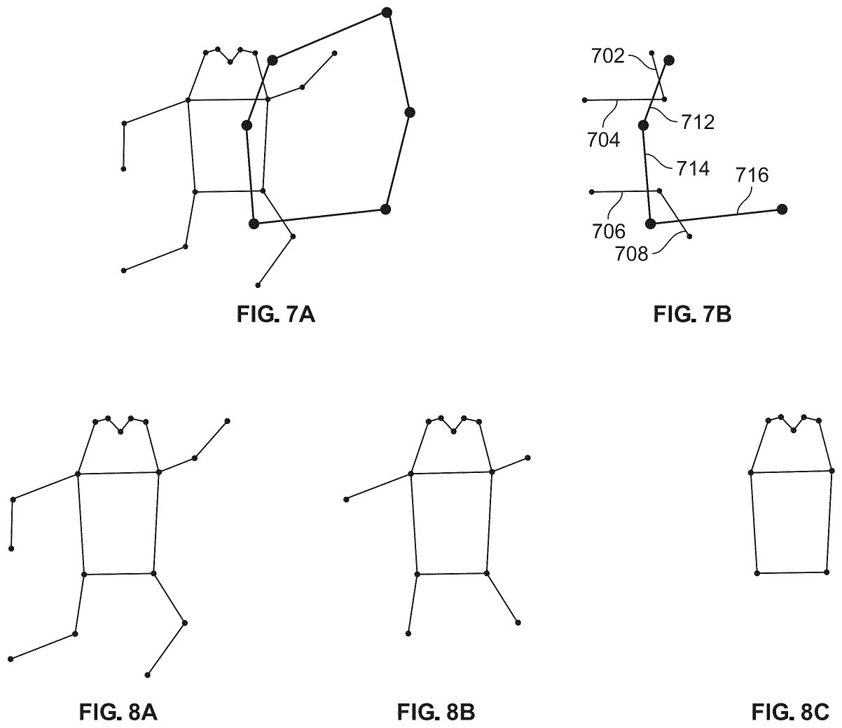

For example, FIG. 7A illustrates a scenario in which certain skeletal segments of a person are determined to have crossed more than one boundary. This is an example of the outside-in tracking that was described earlier with reference to FIG. 3.

Based on determinations such as those described earlier with reference to FIG. 6, the identification of the particular crossings are made. For example, FIG. 7B illustrates four particular crossings or intersections with reference to the scenario illustrated in FIG. 7A. Two crossings (by the skeletal segment 702 and the skeletal segment 704) are with respect to the boundary 712. One crossing (by the skeletal segment 706) is with respect to the boundary 714. One crossing (by the skeletal segment 708) is with respect to the boundary 716.

FIGS. 7A and 7B illustrate an example of outside-in tracking using features described herein with reference to various embodiments. It is understood that such features are similarly applicable toward performance of inside-out tracking.

According to at least one embodiment, if at least one crossing of a boundary is detected, then a notification is sent to the PSM professional. By way of example, a text message may be sent via Short Message Service (SMS) or Multimedia Messaging Service (MMS) to a Provider application used by the PSM personnel. Here, the device may leverage a messaging communication protocol between the device and the Provider application.

According to at least one embodiment, the device sends a notification to a managed backend service (e.g., the same backend service that handles all other communication between the device and the PSM device, including webRTC video feeds). Such a service may be a Call Management System (CMS).

That ‘managed backend’ would then trigger any number of rule-based behaviors for the alert (e.g., logging it in some other system, sending an SMS, etc.).

According to at least one embodiment, an alert appearing on the PSM side may take the form of a flashing light or boundary around the video capturing the patient, to attract the PSM's attention. According to at least one alternative embodiment, the alert may take the form of a light on the desk or any other visual effect.

As described earlier with reference to FIGS. 7A and 7B, the determination regarding crossings of boundaries may be made for all skeletal segments that were identified (e.g., all 18 segments illustrated in FIG. 5).

However, according to various embodiments, such determination may be made for fewer than all skeletal segments that have been defined. For example, such determination may be made for some particular skeletal segments, but not for others.

The above features will be described with reference to sensitivity settings that are selectable by the PSM professional. In at least one embodiment, the PSM professional may select a particular sensitivity setting, concurrent with selecting virtual boundaries (see, e.g., virtual boundaries 108 of FIG. 1). As will be described in more detail below, such a selection effectively selects the set of skeletal segments for which tracking is to be performed.

According to at least one embodiment, such selection may be made from among multiple pre-defined sensitivity settings.

FIG. 8A illustrates an example of a set of skeletal segments corresponding to a highest (or high) sensitivity setting according to at least one embodiment.

Here, all skeletal segments that have been identified are to be tracked. For example, if all 18 segments illustrated in FIG. 5 have been identified, then all of these 18 segments are to be tracked. Accordingly, a detected crossing by any of the 18 skeletal segments would cause a notification to be sent to PSM professionals. In such a setting, tracking of movement of a body of a person (e.g., a patient) would encompass identified segments in the torso, the head, the arms (e.g., from shoulder to wrist), and the legs (e.g., from hip to ankle) of the person.

FIG. 8B illustrates an example of a set of skeletal segments corresponding to a medium (or intermediate) sensitivity setting according to at least one embodiment.

Here, relative to the setting of FIG. 8A, (potentially) fewer than all skeletal segments that have been identified are to be tracked. For example, if all 18 segments illustrated in FIG. 5 have been identified, then fewer than all 18 segments are to be tracked.

For example, with reference to FIG. 8B, in such a setting, tracking of movement of a body of a person (e.g., a patient) would encompass identified segments in the torso, the head, the arms (e.g., from shoulder to elbow), and the legs (e.g., from hip to knee) of the person. However, certain segments further removed from the core of the body (e.g., identified segments in the lower arms and the lower legs of the body of the person) are not to be considered. In this manner, the sensitivity level of the medium sensitivity setting is considered as being lower (e.g., less sensitive) than that of the high sensitivity setting.

FIG. 8C illustrates an example of a set of skeletal segments corresponding to a low (or lowest) sensitivity setting according to at least one embodiment.

Here, relative to the respective settings of FIGS. 8A and 8B, (potentially) fewer than all skeletal segments that have been identified are to be tracked. For example, if all 18 segments illustrated in FIG. 5 have been identified, then fewer than all 18 segments are to be tracked.

For example, with reference to FIG. 8C, in such a setting, tracking of movement of a body of a person (e.g., a patient) would encompass identified segments in the torso and the head of the person. However, certain segments further removed from the core of the body (e.g., identified segments in the upper and lower arms and the upper and lower legs of the body of the person) are not to be considered. In this manner, the sensitivity level of the low sensitivity setting is considered as being lower (e.g., less sensitive) than that of the high sensitivity setting and that of the medium sensitivity setting.

FIGS. 8A, 8B and 8C illustrate example sensitivity settings. However, it is understood that one or more additional sensitivity settings may be defined to encompass other subset(s) of identified segments.

As described earlier, the determination regarding crossings of boundaries may be made for fewer than all skeletal segments that have been defined. Alternatively, although the determination regarding crossings of boundaries are made for all skeletal segments that have been identified (e.g., all 18 segments illustrated in FIG. 5), notifications may be sent to PSM professionals in only particular situations. For example, the notifications may be triggered only in response to detection of boundary crossings by certain skeletal segments (e.g., a subset of all the segments that have been identified).

According to at least one embodiment, frame interpolation and/or smoothing may be used in analyzing video images. Such techniques may be used to address temporal instability. For example, update rejection may be applied towards updates that fall outside the range of realistic values, and to prevent tracked joints that are temporarily occluded or missed by pose detection from disappearing. As another example, smoothing may be used to reduce jitter in situations where a tracked subject is not moving in the video.

According to at least one embodiment, depth data, e.g., provided from a stereo camera pair, may be used to improve system accuracy or reduce the likelihood of scenarios in which a joint (or keypoint) is lost from partial occlusion.

According to at least one embodiment, a second camera may be used to monitor or track movements of a person to address, for example, situations in which the person moves or occludes the primary camera. This increases reliability in detection and resulting transmission of notifications to the PSM professional.

As described earlier with reference to various embodiments, tracking and transmission of notifications may be performed based on video images (e.g., video image 102) captured by one or more cameras that are positioned in a healthcare setting. According to a further embodiment, such features may be performed further based on outputs (e.g., signals) from one or more additional sensors. Such sensors may include a reed switch. For example, a reed switch may be positioned at or around the entry/exit door of the room of a medical patient, in order to detect the opening of the door. Outputs provided by such a sensor may be useful for improving prediction results and/or controlling an importance or urgency of a given notification.

For example, the above features will be described with reference to the following two timeline scenarios. For purposes of illustration, both scenarios refer to a reference time T.

$$T + 0 seconds : door open event detected \qquad Scenario A$$

$$T + 10 seconds : boundary crossing detected$$

$$T + 0 seconds : boundary crossing detected \qquad Scenario B$$

$$T + 10 seconds : door open event detected$$

The same events occur in both scenarios. However, the sequence in which these events occur is different.

Given scenario A, it may be reasonable to deduce that the following has occurred: a nurse or care provider has opened the door to enter the room of the patient, and, ten seconds later, the nurse crosses a boundary to interact with the patient.

Given scenario B, it may be reasonable to deduce that the following has occurred: the patient has crossed a boundary to arise from a corresponding piece of furniture (e.g., bed or chair), and, ten seconds later, the patient opens the door in an attempt to leave the room unassisted.

According to at least one embodiment, both the events of scenario A and the events of scenario B would result in triggering an alert or notification. However, an urgency level of the notification triggered by scenario A may be lower than an urgency level of the notification triggered by scenario B. This is because the potential events underlying scenario B warrant a more urgent response, e.g., to retrieve the patient before he wanders further. As such, the notification triggered by scenario B may include an urgency indicator that would not be present in the notification triggered by scenario A.

As described earlier, a reed switch is an example of a sensor that may be utilized concurrently with one or more cameras that are positioned in a healthcare setting. Other examples of such sensors may include: pressure mats and bed sensors for sensing presence of a person, infrared sensors for sensing body heat and/or movement, wearable devices for sensing patient location, and/or acoustic sensors for triangulating the location of a patient (which may additionally use audio data recorded by the one or more cameras). These sensors could also be integrated with the system's backend and integrated in the PSM monitoring tools

According to at least one embodiment, a sequence of detected events may be stored (or recorded) as an event stream which can be used to ensure compliance with fall management response protocols. For example, in response to detection of a boundary crossing, the device may record an event such as “RAIL_BOUNDARY_CROSSED.” In response to detecting that another person (e.g., a healthcare provider) enters the room to assist the patient, the device may record another detection event such as “ENTER_ROOM_EVENT.” Such events may be captured in an event stream as follows:

$$T = 0 s , “ RAIL\_BOUNDARY \_CROSSED ”$$

$$T = 50 s “ ENTER\_ROOM \_EVENT ”$$

A stream of such events is stored in a time series database or stream analytics (e.g., Kafka, TimeseriesDB, Amazon Simple Queue Service (SQS)), and can be analyzed using business rules to help ensure compliance. By way of example, a particular business rule may be: “A provider must respond to a fall event within 1 minute of detection.” Additionally, data streams can be analyzed using anomaly detection to detect potentially abnormal event sequences that are not yet captured by relevant business rules.

According to at least one embodiment, notifications that are triggered may be provided to more than one recipient. For example, a notification may be provided to a set of recipients that was previously specified by a user. Such recipients may include one or more providers located at or within the immediate surroundings (e.g., the hospital, the nursing home, etc.), or elsewhere. By way of example, a notification concerning a fall of a patient may be sent to one or more systems already employed by the hospital that aggregate events and provide notifications via, e.g., pagers, mobile app notifications, in-Electronic Health Record (HER) notifications, e-mails, or other alert and notification mechanisms outside of devices and/or systems described herein.

According to aspects disclosed herein, PSM personnel are aided in monitoring one or more high-risk situations concurrently with one single tool. Embodiments of the present invention can help in reducing fall risk and flight risk, and also in addressing situations where other people (visitors or healthcare providers) physically interact with the patient.

PSM personnel are afforded flexibility in selecting the desired boundaries. For example, boundaries may be selected to effectively surround a chair that a patient uses, a bed, or any region that is captured in a video image.

According to aspects disclosed herein, detection of movements is performed by actively tracking skeletal features of one or more persons, and identifying crossings of boundaries by specific parts of the human anatomy (e.g., skeletal segments). Accordingly, a probability of false positives is reduced. Such false positives might possibly result from crossing of boundaries by mere shadows that are cast due to nearby light or light that shine through one or more windows. Other false positives include those that may result from motion of one or more objects that lack human skeletal features. Such objects may include human dolls and human dummies.

According to aspects disclosed herein, features associated with edge computing may be utilized. In edge computing, computing and/or data storage are moved closer to the devices that produce the data and the users who consume it. This allows devices in remote locations to process data at the edge of the network, either by the device or a local server.

For example, according to one or more embodiments, computing associated with tracking the movements of human skeletal segments and computation associated with detecting crossings of boundaries are all executed on the edge in the patient's room. This allows for lower computation demands on the PSM side, and allows PSM personnel to run the monitoring software without requiring more powerful computing power on their end. Additionally, this allows for a greater degree of patient privacy as data regarding their movements are not transmitted outside the device for computation.

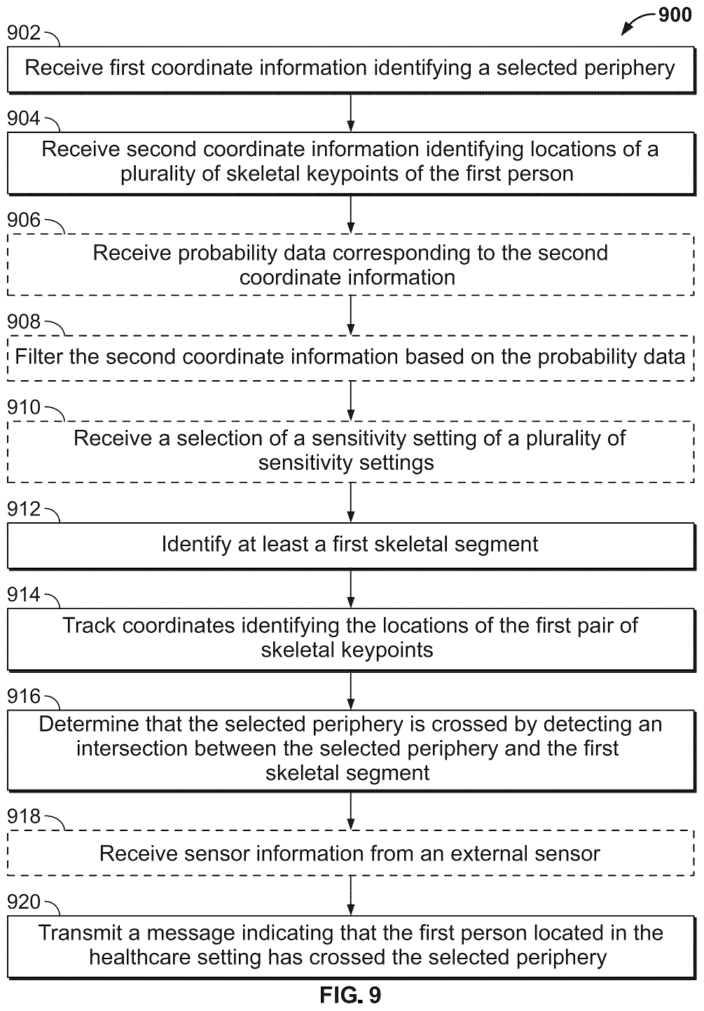

FIG. 9 illustrates a flowchart of a method of monitoring movement of a skeleton of at least a first person located in a healthcare setting according to at least one embodiment.

At block 902, first coordinate information is received. The first coordinate information identifies a selected periphery of a portion of a displayed video image, the displayed video image depicting the healthcare setting.

For example, the selected periphery may be of a polygonal portion of the displayed video image. The first coordinate information may correspond to coordinate information identifying locations of vertices of the polygonal portion.

For example, with reference back to FIG. 1, the first coordinate information identifies virtual boundaries 108 selected to enclose a portion of the video image 102 in which the human patient 104 and the bed 106 are depicted.

At block 904, second coordinate information is received. The second coordinate information identifies locations of a plurality of skeletal keypoints of the first person located in the healthcare setting.

The second coordinate information may be received from an AI model trained to detect the plurality of skeletal keypoints of the first person located in the healthcare setting.

The plurality of skeletal keypoints may include keypoints corresponding to one or more of shoulders, elbows, wrists, hips, knees or ankles of the first person.

For example, with reference back to the silhouette 402 of FIG. 4A, the second coordinate information identifies locations of keypoints such as the shoulders 404a, 404b, the elbows 406a, 406b, the wrists 408a, 408b, the hips 410a, 410b, the knees 412a, 412b, and the ankles 414a, 414b, etc. of the human patient.

At block 906, probability data corresponding to the second coordinate information may be received, concurrent with receiving the second coordinate information.

For example, as described earlier with reference to FIG. 4B, the coordinates of a given keypoint may be accompanied by probability data indicating a level of confidence that the model has with respect to accuracy of the identification of that particular keypoint. By way of example, the probability data (or particular ranges thereof) may indicate high level of confidence, intermediate level of confidence, low level of confidence, etc.

At block 908, the second coordinate information may be filtered based on the probability data.

For example, as described earlier with reference to FIGS. 4B and 5, only keypoints having probability data that meet at least a particular confidence threshold (e.g., a threshold value indicating a high level of confidence) may be kept for further processing. In contrast, keypoints not meeting such a threshold may be disregarded.

At block 910, a selection of a sensitivity setting of a plurality of sensitivity settings may be received.

Each of the plurality of sensitivity settings may correspond to a respective subset of skeletal segments. For example, as described earlier with reference to FIGS. 8A, 8B and 8C, a first sensitivity setting may correspond to skeletal segments in a human torso, head, upper arms, lower arms, upper legs and lower legs, a second sensitivity setting may correspond to skeletal segments excluding skeletal segments in the lower arms and the lower legs, and a third sensitivity setting may correspond to skeletal segments excluding skeletal segments in the upper arms, the lower arms, the upper legs and the lower legs.

At block 912, based on the second coordinate information, at least a first skeletal segment is identified. The first skeletal segment is defined by a first pair of skeletal keypoints of the plurality of skeletal keypoints.

For example, description regarding the “upper left arm” segment 502 was provided earlier with reference to FIG. 6.

At block 914, coordinates identifying the locations of the first pair of skeletal keypoints are tracked over a plurality of successive video images depicting the healthcare setting.

For example, as described earlier, with reference to FIG. 6, coordinates identifying the locations of skeletal keypoints defining the “upper left arm” segment 502 are tracked.

Tracking coordinates identifying the locations of the first pair of skeletal keypoints may be performed based on the selected sensitivity setting of block 910.

For example, tracking coordinates identifying the locations of the first pair of skeletal keypoints may be performed based on the first skeletal segment being included in the subset of skeletal segments corresponding to the selected sensitivity setting. In contrast, tracking coordinates identifying the locations of the first pair of skeletal keypoints is not performed based on the first skeletal segment not being included in the subset of skeletal segments corresponding to the selected sensitivity setting.

At block 916, based on the first coordinate information, it is determined that the selected periphery is crossed by detecting an intersection between the selected periphery and the first skeletal segment.

For example, as described earlier with reference to FIG. 6, an intersection between the “upper left arm” segment 502 and a particular boundary 602 of the closed polygon at a particular moment in time (i.e., in a particular video image or video frame) is detected.

For example, the detected intersection may be between the first skeletal segment and an edge of the selected periphery (see, e.g., block 902) defined by two adjacent vertices of the polygonal portion.

At block 918, sensor information may be received from an external sensor located in the healthcare setting.

For example, outputs (e.g., signals) may be received from one or more additional sensors such as a reed switch. For example, a reed switch may be positioned at or around the entry/exit door of the room of a medical patient, in order to detect the opening of the door.

At block 920, in response to determining that the selected periphery is crossed, a message indicating that the first person located in the healthcare setting has crossed the selected periphery is transmitted.

For example, as described earlier with reference to FIGS. 7A and 7B, if at least one crossing of a boundary is detected, then a notification is sent to the PSM professional. By way of example, a text message may be sent via SMS or MMS to a Provider application used by the PSM personnel. Here, the device may leverage a messaging communication protocol between the device and the Provider application.

The transmitted message may include an urgency indication based on the received sensor information (of block 918) and the detected intersection between the selected periphery and the first skeletal segment. For example, the urgency indication may be based on a relative timing between the received sensor information and the detected intersection.

FIG. 10 is a block diagram of an AI device 1000 (or apparatus) according to at least one embodiment to at least one embodiment.

Referring to FIG. 10, the AI device 1000 may be operated by a PSM professional, as described earlier with reference to FIG. 1. The AI device 1000 is configured to perform features described herein with respect to monitoring movement of a skeleton of at least one person located in a healthcare setting (see, e.g., FIG. 9).

The AI device 1000 may include a memory 1004, a processor 1006, and a power supply 1002, and the processor 1006 may further include an AI processor 1008. The processor 1006 may be referred to as a main controller. The AI device 1000 may also include a camera 1010, a microphone 1012, and a speaker 1014. The camera 1010 may capture images including images of the room of a medical patient (see, e.g., video image 102 of FIG. 1).

The AI device 1000 may include an interface 1016. The interface can be configured using at least one of a communication unit (transceiver), a terminal, a pin, a cable, a port, a circuit, an element or a device.

The memory 1004 is electrically connected with the processor 1006. The memory 1004 can store data processed in the processor 1006. With regards to hardware configuration, the memory 1004 may be configured using at least one of a ROM, a RAM, an EPROM, a flash drive, or a hard drive. Also, the memory 1004 may store instructions, that, when executed, cause the processor 1006 to perform features described herein (see, e.g., FIG. 9). The memory 1004 can store various types of data for the overall operation of the AI device 1000, such as a program for processing or control of the processor 1006. The memory 1004 may be integrated with the processor 1006. In one or more particular embodiments, the memory 1004 may be classified as a lower configuration of the processor 1006.

The power supply 1002 can supply power to the AI device 1000. The power supply 1002 can be provided with power from a power source (e.g., a battery) included in the AI device 1000 and can supply the power to each module of the AI device 1000.

The processor 1006 can be electrically connected to the memory 1004, the interface 1016, and the power supply 1002 and exchange signals with these components. The processor 1006 can be realized using at least one of application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, and electronic units for executing other functions.

The processor 1006 can be operated by power supplied from the power supply 8190. The processor 1006 can receive data, process the data, generate a signal, and provide the signal while power is supplied thereto by the power supply 1002.

The processor 1006 can receive information from devices connected with the AI device 1000. The processor 1006 can provide control signals to devices connected with the AI device 1000 through the interface 1016.

As described earlier, the processor 1006 may include an AI processor 1008. The AI processor 1008 may operate AI models including one or more analytical models that have been described herein with reference to various embodiments. For example, the one or more analytical models may analyze one or more video images of a video feed, as described earlier with reference to FIGS. 4A and 4B.

The AI device 1000 may include at least one printed circuit board (PCB). The memory 1004, the interface 1016, the power supply 1002, and the processor 1006 may be electrically connected to the PCB.

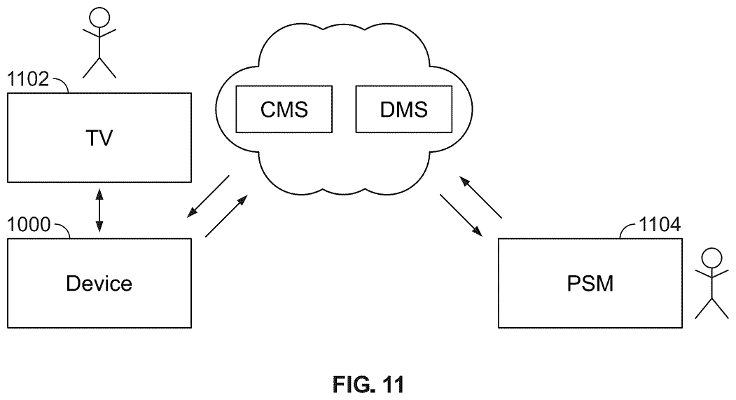

FIG. 11 is a diagram illustrating a system in which an AI device (e.g., AI device 1000 of FIG. 10) and devices operable by one or more PSM professionals are linked according to at least one embodiment.

For example, the AI 1000 is connected to TV 1102 (e.g., display device) operable by a first PSM professional. As described earlier with reference to FIG. 1, the display device (e.g., a video monitor) is utilized by the first PSM professional. Based on the display of a video image (e.g., video image 102 of FIG. 1), the PSM professional may operate a visual computer-implemented tool implemented in the device 1000 to define one or more virtual boundaries 108.

According to at least one embodiment, communications between the display device 1102 and the AI device 1000 are exchanged via Internet, Ethernet, etc. rather than via a cloud-based system. This aids in addressing concerns relating to privacy and/or latency. Similarly, according to at least one embodiment, models running on the AI device (see, e.g., AI processor 1108) are running “on target” and not remotely in a cloud-based system. This also aids in addressing concerns relating to privacy and/or latency.

If at least one crossing of a boundary is detected, then a notification is sent to a device 1104 operable by a second PSM professional. By way of example, a text message may be sent via SMS or MMS to a Provider application used by the second PSM personnel. The notification may also be sent via a cloud-hosted backend (e.g., Call Management System (CMS), DMS). According to at least one embodiment, the CMS and DMS are located on-premises (e.g., in the hospital or within the hospital network). This would improve latency and privacy if the backend systems are inside the same network as the second PSM professional.

According to at least one embodiment, a direct connection between patient-side device and PSM computers is utilized instead of a cloud-based infrastructure.

The above-described present disclosure can be implemented with computer-readable code in a computer-readable medium in which program has been recorded. The computer-readable medium may include all kinds of recording devices capable of storing data readable by a computer system. Examples of the computer-readable medium may include a hard disk drive (HDD), a solid state disk (SSD), a silicon disk drive (SDD), a ROM, a RAM, a CD-ROM, magnetic tapes, floppy disks, optical data storage devices, and the like and also include such a carrier-wave type implementation (for example, transmission over the Internet). Therefore, the above embodiments are to be construed in all aspects as illustrative and not restrictive. The scope of the invention should be determined by the appended claims and their legal equivalents, and not by the above description, and all changes coming within the meaning and equivalency range of the appended claims are intended to be embraced therein.

What is claimed is:

1. A method of monitoring movement of a skeleton of at least a first person located in a healthcare setting, the method comprising:
receiving first coordinate information identifying a selected periphery of a portion of a displayed video image, the displayed video image depicting the healthcare setting;
receiving second coordinate information identifying locations of a plurality of skeletal keypoints of the first person located in the healthcare setting;
based on the second coordinate information, identifying at least a first skeletal segment, the first skeletal segment defined by a first pair of skeletal keypoints of the plurality of skeletal keypoints;
tracking coordinates identifying the locations of the first pair of skeletal keypoints over a plurality of successive video images depicting the healthcare setting;
based on the first coordinate information, determining that the selected periphery is crossed by detecting an intersection between the selected periphery and the first skeletal segment; and
in response to determining that the selected periphery is crossed, transmitting a message indicating that the first person located in the healthcare setting has crossed the selected periphery.

2. The method of claim 1, wherein:
the selected periphery is of a polygonal portion of the displayed video image; and
the first coordinate information corresponds to coordinate information identifying locations of vertices of the polygonal portion.

3. The method of claim 2, wherein the detected intersection is between the first skeletal segment and an edge of the selected periphery defined by two adjacent vertices of the polygonal portion.

4. The method of claim 1, further comprising:
concurrent with receiving the second coordinate information, receiving probability data corresponding to the second coordinate information; and
filtering the second coordinate information based on the probability data.

5. The method of claim 1, wherein the second coordinate information is received from an artificial intelligence (AI) model trained to detect the plurality of skeletal keypoints of the first person located in the healthcare setting.

6. The method of claim 1, wherein the plurality of skeletal keypoints of the first person located in the healthcare setting include keypoints corresponding to one or more of shoulders, elbows, wrists, hips, knees or ankles of the first person.

7. The method of claim 1, further comprising:
receiving a selection of a sensitivity setting of a plurality of sensitivity settings,
wherein tracking coordinates identifying the locations of the first pair of skeletal keypoints is performed based on the selected sensitivity setting.

8. The method of claim 7, wherein:
each of the plurality of sensitivity settings corresponds to a respective subset of skeletal segments; and
tracking coordinates identifying the locations of the first pair of skeletal keypoints is performed based on the first skeletal segment being included in the subset of skeletal segments corresponding to the selected sensitivity setting.

9. The method of claim 8, wherein tracking coordinates identifying the locations of the first pair of skeletal keypoints is not performed based on the first skeletal segment not being included in the subset of skeletal segments corresponding to the selected sensitivity setting.

10. The method of claim 8, wherein the plurality of sensitivity settings comprises:
a first sensitivity setting corresponding to skeletal segments in a human torso, head, upper arms, lower arms, upper legs and lower legs;
a second sensitivity setting corresponding to skeletal segments excluding skeletal segments in the lower arms and the lower legs; and
a third sensitivity setting corresponding to skeletal segments excluding skeletal segments in the upper arms, the lower arms, the upper legs and the lower legs.

11. The method of claim 1, further comprising:
receiving sensor information from an external sensor located in the healthcare setting,
wherein the transmitted message includes an urgency indication based on the received sensor information and the detected intersection between the selected periphery and the first skeletal segment.

12. The method of claim 11, wherein the urgency indication is based on a relative timing between the received sensor information and the detected intersection.

13. An artificial intelligence (AI) device configured to monitor movement of a skeleton of at least a first person located in a healthcare setting, the AI device comprising:
at least one transceiver; and
at least one processor configured to:
receive first coordinate information identifying a selected periphery of a portion of a displayed video image, the displayed video image depicting the healthcare setting;
receive second coordinate information identifying locations of a plurality of skeletal keypoints of the first person located in the healthcare setting;
based on the second coordinate information, identify at least a first skeletal segment, the first skeletal segment defined by a first pair of skeletal keypoints of the plurality of skeletal keypoints;
track coordinates identifying the locations of the first pair of skeletal keypoints over a plurality of successive video images depicting the healthcare setting;
based on the first coordinate information, determine that the selected periphery is crossed by detecting an intersection between the selected periphery and the first skeletal segment; and
in response to determining that the selected periphery is crossed, transmit a message indicating that the first person located in the healthcare setting has crossed the selected periphery.

14. A non-transitory storage medium storing instructions that, when executed, cause at least one processor to perform operations, the operations comprising:
receiving first coordinate information identifying a selected periphery of a portion of a displayed video image, the displayed video image depicting a healthcare setting;
receiving second coordinate information identifying locations of a plurality of skeletal keypoints of a first person located in the healthcare setting;
based on the second coordinate information, identifying at least a first skeletal segment, the first skeletal segment defined by a first pair of skeletal keypoints of the plurality of skeletal keypoints;
tracking coordinates identifying the locations of the first pair of skeletal keypoints over a plurality of successive video images depicting the healthcare setting;
based on the first coordinate information, determining that the selected periphery is crossed by detecting an intersection between the selected periphery and the first skeletal segment; and
in response to determining that the selected periphery is crossed, transmitting a message indicating that the first person located in the healthcare setting has crossed the selected periphery.
